(19) United States
(12) Patent Application Publication
(10) Pub. No.: US 2006/0229541 A1
(43) Pub. Date: Oct. 12, 2006

(54) Orthopedic inlay

(76) Inventors: Andreas Hassler, Rohrdorf (DE); Gero Hopmann, Neubiberg (DE)

Correspondence Address: REED SMITH LLP, 3110 FAIRVIEW PARK DRIVE, SUITE 1400, FALLS CHURCH, VA, 22042, US

(21) Appl. No.: 10/542,707

(22) PCT Filed: Jan. 20, 2004
(86) PCT No.: PCT/EP04/00398

§ 371 Date: May 9, 2006

(30) Foreign Application Priority Data
Jan. 28, 2003 (DE) 103 03 327.0

(51) Int. Cl. A61F 5/00; A61F 13/06; A61F 13/00

(52) U.S. Cl. 602/27; 602/23; 602/65

(57) ABSTRACT

Orthotic inlay (59) for arrangement in a supporting shell configuration of an orthotic device for a body part having a molded cushion (10) for shaping to the body part with at least one cushion chamber designed in an airtight casing, said chamber being evacuable via a valve mechanism (48) and having a molded body filling consisting of a plurality of molded bodies and having a molded cushion casing (49) to accommodate the molded cushion, whereby the molded cushion (10) has a planar cushion blank, and after being accommodated in the respective receptacle areas of the molded cushion casing side parts (28, 29) and a strap part (16) of the molded cushion form a shell-shaped body part receptacle for enclosing at least a portion of the body part that is to be accommodated.

The present invention relates to an orthotic inlay for arrangement in a supporting shell configuration of an orthotic device for a body part having a molded cushion for molding to the body part, with at least one cushion chamber formed in an airtight casing, said chamber being evacuable through a valve mechanism and having a molded body filling consisting of a plurality of molded bodies and having a molded cushion casing to accommodate the molded cushion.

In addition, this invention relates to a molded cushion for forming an orthotic inlay, a molded cushion casing for forming an orthotic inlay and a valve mechanism for such a molded cushion.

Orthotic inlays of the type defined in the introduction serve for lining the inside of shell configurations used for example for the treatment of fractures of the lower leg and in particular for the treatment of a ruptured Achilles tendon. Such orthotic inlays may also be used for fractures of the forearm or for support or immobilization of the cervical vertebrae. The molded cushion then has a lining and cushioning function. In addition, it should be possible to introduce the supporting forces which are applied by the outer supporting shell configuration in the most direct way possible into the respective body part, i.e., the lower leg, for example. Such molded cushions are therefore equipped with an airtight casing which serves to develop a cushion chamber that is equipped with a molded body filling consisting of a plurality of molded bodies. After completely wrapping the molded cushion around the lower leg, the cushion chamber is evacuated by means of a valve mechanism provided in it, thereby immobilizing the molded bodies and thus securing the relative arrangement of the molded bodies of the molded cushion achieved through this wrapping.

It is clear from the preceding discussion that careful fitting of the molded cushion, e.g., to the contour of the lower leg, i.e., the patient's calf and foot, is extremely important, because the relative positioning thereby achieved is retained due to the evacuation procedure and subsequently determines the efficacy of the action of the supporting forces on the lower leg by the shell configuration. Since the molded cushion is fitted to the contour of the lower leg starting from a planar cushion blank of the molded cushion, the fitting of the planar cushion blank to the contour of the respective body part therefore requires a great deal of skill accordingly. The same thing is also true of performing the actual evacuation process, which must be repeated many times in some cases to make corrections in the seating of the cushion, which is associated with repeated operation of the valve mechanism accordingly.

Therefore, the object of the present invention is to permit simplified handling of an orthotic inlay of the type defined above.

This object is achieved by an orthotic inlay having the features of claim 1.

With the inventive orthotic inlay, the molded cushion has a planar cushion blank with a base part with at least two side parts and a strap part separated from the base part by constriction. The molded cushion casing has at least three receptacle areas, one each assigned to the side parts of the base part and the strap part, namely two side part receptacles and one strap part receptacle which are arranged adjacent to one another such that after being accommodated in the respective receptacle areas of the molded cushion casing, the side parts and the strap part of the molded cushion form a shell shape body part receptacle for at least partial sheathing of the body part to be accommodated.

Thus the development of a shell-shaped body part receptacle is made possible already in the vented state of the molded cushion in cooperation with the molded cushion casing, so that this already provides a “rough fitting” to the contour of the body part, greatly facilitating a subsequent precision “fine fitting” by evacuation of the molded cushion. Here the constriction acts as a hinge area on the one hand. On the other hand, this also prevents unwanted accumulation of molded bodies in one area and a shortage of molded bodies in an adjacent area.

The cushion blank of an orthotic inlay especially suitable for arrangement on the lower leg may be designed to be T-shaped in a foot part with a sole part provided for the sole of the foot and a heel part that is divided from the sole part by a heel constriction and runs across the longitudinal axis of the sole part with foot side parts extending on both sides of the longitudinal axis.

The T-shaped design of the foot part permits a simple means of converting the foot part into a basic configuration in which only relatively minor deformation of the foot side straps is necessary to fit it to the contour of the foot to achieve optimum seating of the foot part; this is accomplished by pivoting the foot side straps about the longitudinal axis of the sole part and/or the calf part and pivoting the foot sole part about the axis of the heel constriction.

If according to a preferred embodiment, the foot side parts have lower edges running essentially at a right angle to the longitudinal axis of the sole part and upper edges running to the free side part ends with an inclination toward the lower edges, then the relative positioning of the foot side parts in relation to the foot sole part and the fitting of the foot side parts to the contour of the foot are further simplified.

It has an especially positive effect on the simple deformability of the foot side parts for fitting to the sides of the foot if the cushion chamber is designed like a frame in the area of the foot side parts with a recess surrounded on all sides by the cushion chamber.

A facilitated adjustment of the molded cushion to the contour of the heel and/or the ankle joint is made possible if the heel part is provided with a recess in the area of the heel.

Conforming to the shape of the calf when applying the molded cushion is facilitated when the cushion chamber has a U-shaped cushion area in the area of the calf part with a chamber base and two chamber legs, a leg connection being provided between the chamber legs.

A particularly effective and at the same time simple design of a molded cushion casing to achieve a shaping effect that acts on the molded cushion is made possible when the molded cushion casing has connecting seams joining a bottom layer and a top layer together to design and separate the receptacle areas.

Due to the arrangement of the molded cushion in the suitably shaped molded cushion casing, the molded cushion is converted on the whole into a basic configuration on the basis of which only a relatively minor deformation of the molded cushion is necessary for complete fitting of the molded cushion to the body part contour.

In the case of the design of an orthotic inlay for arrangement on the lower leg, an especially extensive fitting of the foot part of the molded cushion to the contour of the foot is made possible if the molded cushion casing has a design of the foot receptacle part in which a sole pocket to accommodate the sole part is provided with two essentially parallel side part pockets running adjacent to the former to accommodate the foot side straps in addition to a calf part receptacle and a foot part receptacle to accommodate the calf part and the foot part of the molded cushion.

Especially simple handling of the molded cushion and thus the orthotic insert is supported by a valve mechanism which has a valve connection made of an elastically deformable plastic material for attaching to the airtight casing and a valve insert accommodated in the valve connection to accommodate a membrane disk sealing the valve cross section of the valve insert, whereby a clamping ring displaceable in the longitudinal direction of the valve connection is provided on the valve connection and can be converted from a sealing position, in which the membrane disk is essentially in flat contact with a sealing edge of the valve seating, into a venting position in which the membrane disk is deformed by the deformed valve connection.

The inventive design of the valve mechanism permits simple one-handed operation of the valve mechanism in which only axial displacement of a clamping ring is necessary to operate the valve mechanism. This makes it possible to combine the operation of the valve mechanism with a process of attaching a hose connection to the vacuum pump device on the valve connection without requiring the valve mechanism to be switched before establishing the hose connection.

If the valve insert with this valve mechanism is also designed in the form of a sleeve with a membrane guide for accommodating a guidance journal of the membrane disk arranged concentrically with the sealing edge of the valve insert, then a high operating reliability of the valve mechanism is achieved with a comparatively simple design.

A preferred embodiment of the orthotic inlay is explained in greater detail below with reference to the drawings, which show:

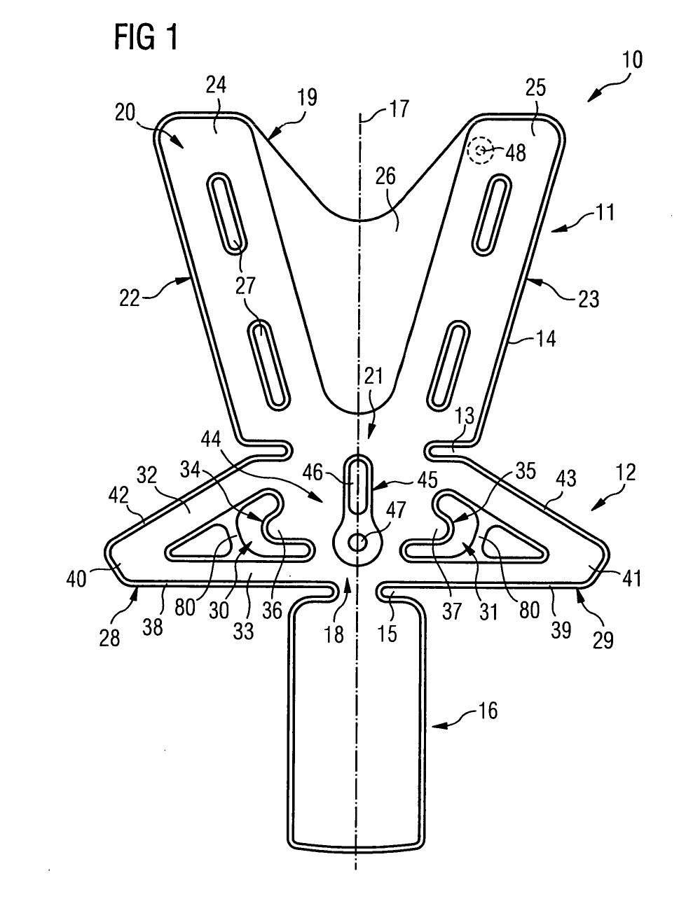

FIG. 1 a cushion blank of a molded cushion spread out in a plane;

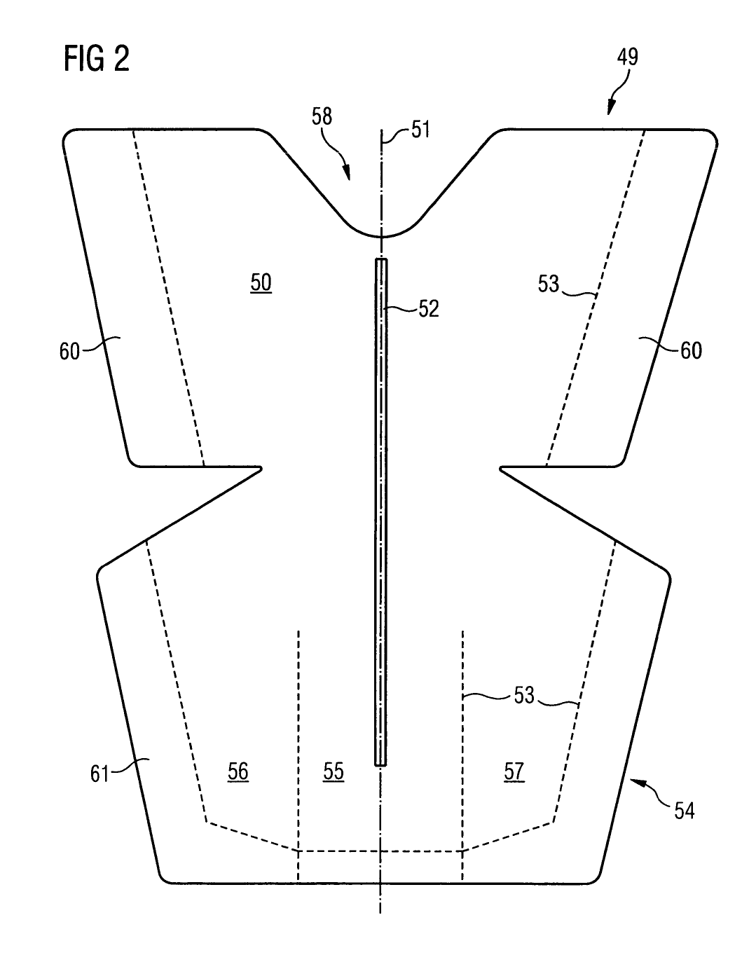

FIG. 2 a molded cushion casing to accommodate the molded cushion depicted in FIG. 1;

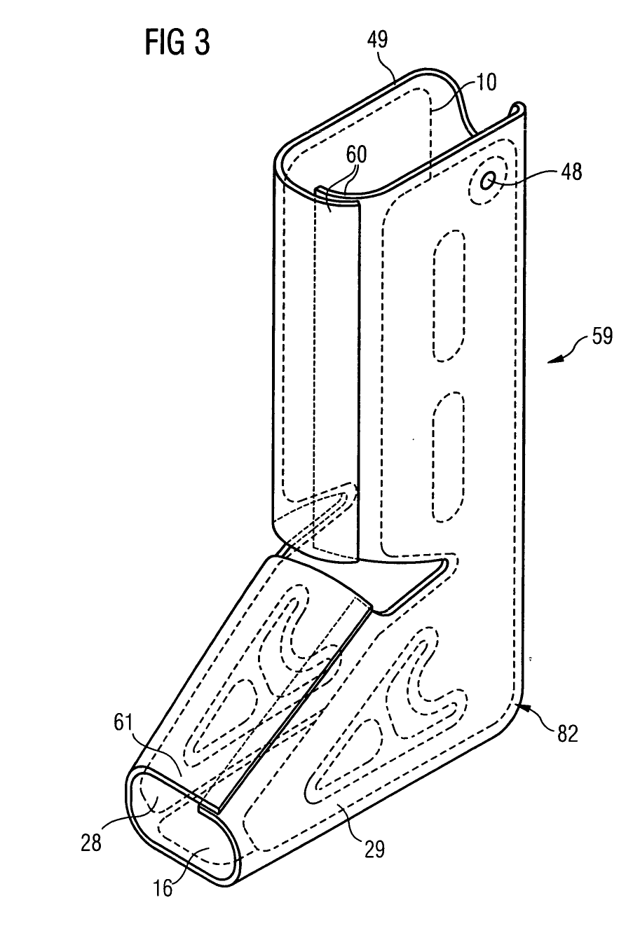

FIG. 3 an orthotic insert formed by accommodating the molded cushion in the molded cushion casing;

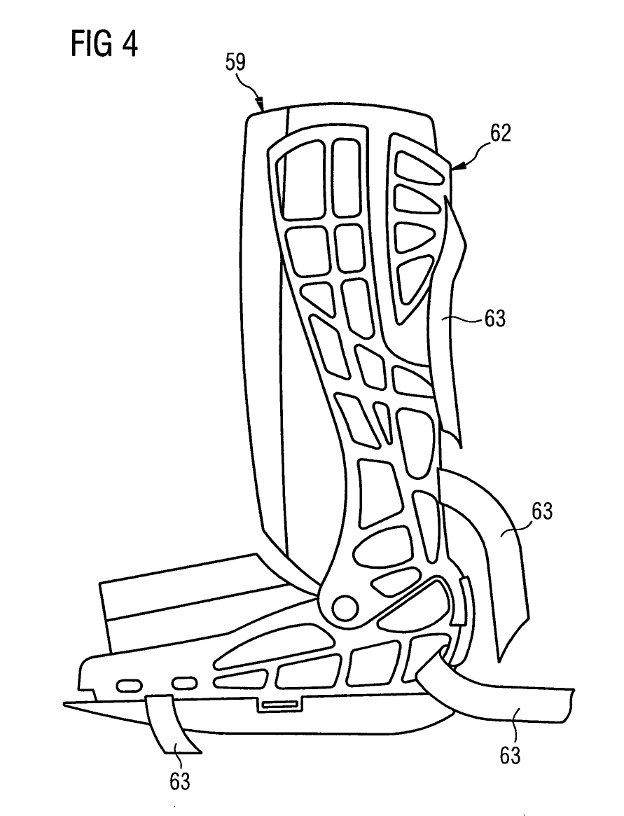

FIG. 4 a shell arrangement supplemented by the orthotic inlay in an application configuration;

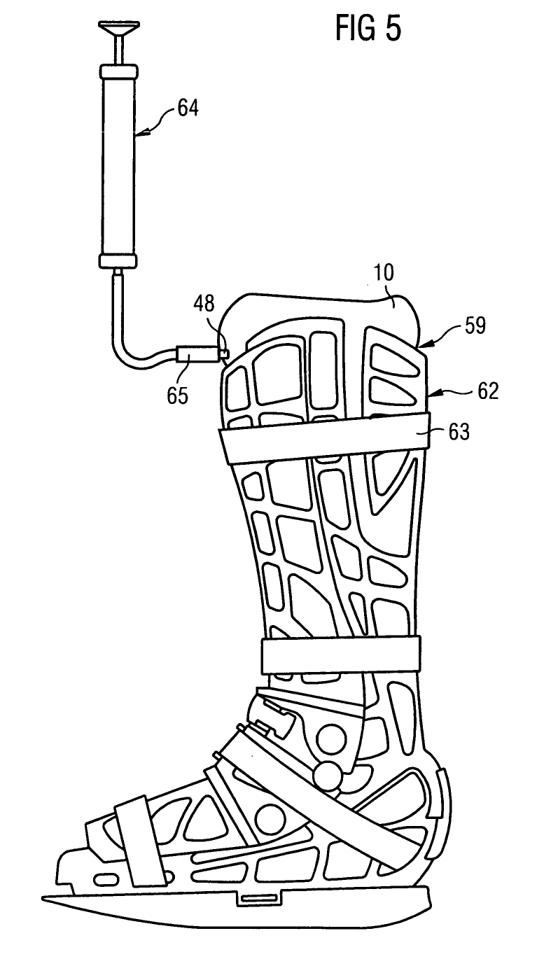

FIG. 5 the shell arrangement provided with the orthotic inlay in the supporting configuration;

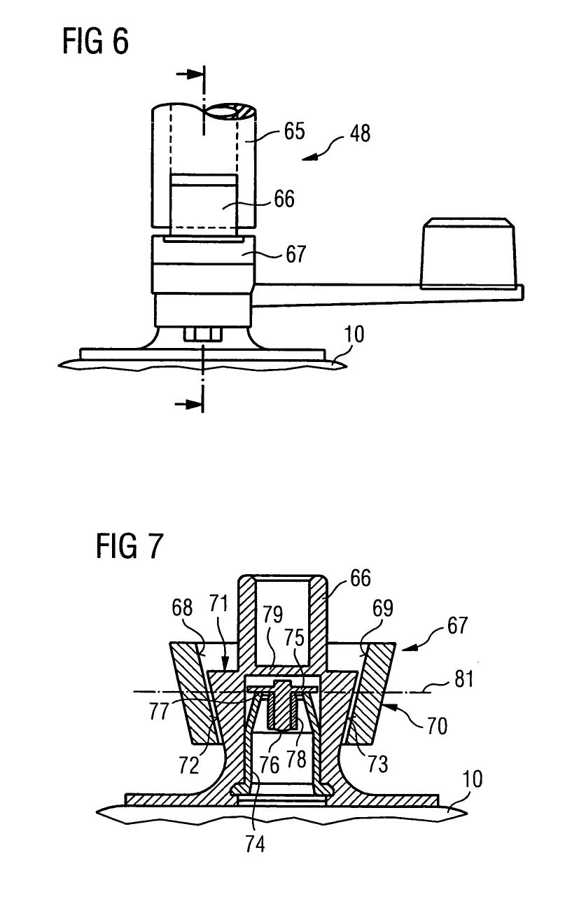

FIG. 6 a valve mechanism of the molded cushion in a side view;

FIG. 7 the valve mechanism shown in FIG. 6, shown here in a sectional view.

FIG. 1 shows a molded cushion 10 with a calf part 11 and a foot part 12 as a planar cushion blank. The calf part 11 is divided from the T-shaped foot part 12 by a first constriction 13 into a cushion contour 14. The foot part 12 designed with a T shape is in turn subdivided by another constriction, referred to below as the heel constriction 15, into a sole part 16, which is arranged in the direction of and symmetrically with a longitudinal axis 17 of the molded cushion, and a heel part 18, which runs across the sole part 16 and is also symmetrical with the longitudinal axis 17.

The molded cushion 10 shown as a flat cushion blank in FIG. 1 consists of an airtight casing 19 formed in the present case by two layers of thermoplastic material and welded in surface areas and along the contour 14 of the cushion to form an airtight cushion chamber 20. In the area of the calf part 11, the cushion chamber 20 is designed in a U shape with a chamber base 21 and in the present case chamber legs 22, 23 diverging from the chamber base 21 and forming essentially a V shape in relation to one another. Between the two chamber legs 22, 23 a connecting area 24, which has a concave contour in the area of the free leg ends 24, 25 of the chamber legs 22, 23, is formed by the plastic layers joined together over the surface area, as explained above. The chamber legs 22, 23 are provided with recesses 27 to achieve the best possible fit and ventilation when the calf part 11 of the molded cushion 10 is applied to a patient's lower leg.

The heel part 18 of the foot part 12 has two foot side parts 28, 29 which extend laterally away from the longitudinal axis 17 of the molded cushion 10, each foot side part having a cushion recess 30, 31 which is surrounded by a frame-like area of the cushion chamber 20. For relative stabilization of the chamber legs 32, 33 of the foot side parts 28, 29 arranged in a V shape in relation to one another, the chamber legs 32, 33 are each connected to connecting webs 80 over the respective cushion recess 30, 31. In addition, an ankle edge area 34, 35 of the cushion recess 30, 31 assigned to the ankle and facing the longitudinal axis 17 is designed with a convex shape to form a chamber tongue 36, 37 protruding into the cushion recess 30, 31. In the present case, the foot side parts 28, 29 have lower edges 38, 39 running approximately perpendicular to the longitudinal axis 17 and have upper edges 42, 43 running to the free side end parts 40, 41 with an inclination toward the lower edges 38 and/or 39. Another cushion recess 45 is provided in a heel area 44 joining the two foot side parts 28, 29 together, said cushion recess having an elongated hole area 46 with a round hole area 47 which begins at the lower end of the elongated hole area 46 and is broadened with respect to the elongated hole area 46. The cushion recess 45 is thus configured according to the accommodation of the calcaneal protuberance of the ankle with the Achilles tendon area adjacent thereto.

The sole part 48 of the foot part 12, designed approximately as a rectangle, is divided by the heel constriction 15 and extends from the heel area 44 of the foot part 12 in the direction of the longitudinal axis 17.

As is also shown clearly in FIG. 1, in the present case the cushion chamber 20 is designed as a chamber having a continuous design over the various areas of the molded cushion 10, said chamber being vented and aerated through only one valve mechanism 48 which is arranged on the leg end 25 of the chamber leg 23 of the calf part 11. For fixation of the adjusted shape of the molded cushion 10 after fitting of the molded cushion 10 to the patient's lower leg, the cushion chamber 20 is provided with a molded body filling (not shown in detail here) consisting of a plurality of molded bodies in a known manner such that after evacuation of the cushion chamber 20 through the valve mechanism 48, the molded bodies remain in their relative positions achieved as a result of the fitting.

FIG. 2 shows a molded cushion casing 49 which is used to accommodate the molded cushion depicted in FIG. 1. In the present case, the molded cushion casing 49 is formed by two layers of textile fabric stitched together, but only the front layer of textile 50 is discernible in the diagram in FIG. 2, this layer being provided along a longitudinal axis 51 with an insertion slot 52 that can be closed for example by means of a zipper (not shown in detail here). The rear textile layer and the front textile layer 50 discernible in FIG. 2 each have a different blank shape which is selected so that, in a configuration which accommodates the lower leg and in which the molded cushion casing 49 is arranged essentially so that it encloses the patient's lower leg, the molded cushion casing 49 is free of stresses in both the outer textile layer and the inner textile layer. In addition, the molded cushion casing 49 depicted in FIG. 2 has stitched areas formed by connecting seams 53 created between the textile layers and serving in a foot part receptacle 54 of the molded cushion casing 49 to create a sole pocket 55 and two side part straps 56, 57 each being arranged on the side of the sole pocket 55 and extending essentially parallel to it.

If the molded cushion shown in FIG. 1 is introduced into the molded cushion casing 49 through the insertion slit 52 in such a way that the calf part 11 of the molded cushion 10 is arranged in a calf part receptacle 58 situated above the foot part receptacle 54 and the foot part 12 of the molded cushion 10 is arranged in the foot part receptacle 54 of the molded cushion casing 49 whereby the sole part 16 is inserted into the sole pocket 55 and the foot side parts 28, 29 are inserted into the side part pockets 56 and 57, then this results approximately in the lower leg receptacle configuration of the molded cushion 10 depicted in FIG. 3 with a shell-shaped foot receptacle area 82 because of the overall shape of the blank of the molded cushion casing 49 and the pockets arranged essentially parallel to one another, namely side part pockets 56, 57 and sole pocket 55. The molded cushion 10 together with the molded cushion casing 49 thus forms an orthotic inlay 59. Here stitched edge strips 60 and 61 limiting the calf part receptacle 58 and the foot part receptacle 54 may be arranged so that they at least partially overlap.

FIG. 4 shows the orthotic inlay 59, which is arranged in the lower leg receptacle configuration and is inserted into a shell arrangement 62 that serves to securely accommodate the patient's lower leg. The shell arrangement 62 which is supplemented by the orthotic inlay 59 may then be arranged on the patient's lower leg and secured in this relative position by closing the closure strips 63 provided for this purpose.

In the supporting configuration of the shell arrangement 62, which is closed by the closure strips 63, as illustrated in FIG. 5, the molded cushion 10 is evacuated through a vacuum pump 64 connected to the valve mechanism 48 of the molded cushion 10. As a result of evacuation of the molded cushion 10, the relative arrangement of the molded bodies (not shown in detail) of the molded body filling achieved by the process of applying the molded cushion 10 is secured so that optimum seating of the shell arrangement 62 on the lower leg is made possible.

This permits the simplest possible handling of the valve mechanism 48 depicted in FIGS. 6 and 7 due to the fact that a clamping ring 67 is pushed into a vent position by pushing a hose mouthpiece 65 of the vacuum pump 64 onto a valve connection 66 of the valve mechanism 48; in this position, the molded cushion 10 can be evacuated by the vacuum pump 64.

As shown in FIG. 7, the clamping ring 67 is provided with a wedge profile 70 which has two clamping ring wedge surfaces 68, 69 and cooperates with a wedge profile 71 on the valve connection 66, said wedge profile having two connection wedge surfaces 72, 73 assigned to the clamping ring wedge surfaces 68, 69 in a complementary manner.

A valve insert 74 which is used to accommodate a membrane disk 75 is situated in the valve connection 66. The membrane disk is axially displaceable with a guidance journal 76 so that it is guided in a membrane guide 78 designed on a sealing edge 77 of the valve insert 74. In the sealing position shown in FIG. 7, the membrane disk 75 is in sealing contact with the sealing edge 77 due to a vacuum created in the interior of the molded cushion 10 and thus also serves as a nonreturn valve during evacuation. In this position, it is possible to move the membrane disk 75 out of the membrane guide 78 due to a vacuum acting through the vacuum pump 64 on the membrane disk 75 until the membrane disk is in contact with a stop web 79 formed in the valve connection 66 to generate or increase the vacuum in the molded cushion 10.

When the clamping ring 67 is moved axially upward out of the sealing position shown in FIG. 7 and into the ventilation position, the clamping ring wedge surfaces 68, 69 of the clamping ring 67 exert a clamping force on the connection wedge surfaces 72, 73 of the valve connection 66 with the result that the valve connection 66, which is made of an elastic plastic material, undergoes deformation in the area of a membrane plane 81 in which the membrane disk 75 is situated so that due to the resulting reduction in cross section of the valve connection 66, the membrane disk 75 is deformed out of the plane 80 of the membrane and there is no longer sealing contact of the membrane disk 75 with the sealing edge 77 of the valve insert 74. Therefore ambient air can flow from the outside into the molded cushion 10 to vent the molded cushion 10.

1. An orthotic inlay for arrangement in a supporting shell configuration of an orthotic device for a body part having a molded cushion for shaping to the body part, with at least one cushion chamber formed in an airtight casing, said chamber being evacuable via a valve mechanism and having a molded body filling consisting of a plurality of molded bodies and having a molded cushion casing to accommodate the molded cushion, characterized in that
the molded cushion has a planar cushion blank with a base part with at least two side parts and a strap part which is separated from the base part by a constriction, and the molded cushion casing has at least three receptacle areas assigned to the side parts of the base part and the strap part, namely two side part receptacles and one strap part receptacle, which are arranged adjacent to one another such that after being accommodated in the respective receptacle areas of the molded cushion casing the side parts and the strap part of the molded cushion form a shell-like body part receptacle for enclosing at least a portion of the body part to be accommodated.

2. A molded cushion for forming an orthotic inlay for arrangement on a lower leg, characterized in that
the molded cushion has a planar cushion blank with a base part with at least two side parts and a strap part which is separated from the base part by a constriction, and the molded cushion casing has at least three receptacle areas assigned to the side parts of the base part and the strap part, namely two side part receptacles and one strap part receptacle, which are arranged adjacent to one another such that after being accommodated in the respective receptacle areas of the molded cushion casing the side parts and the strap part of the molded cushion form a shell-like body part receptacle for enclosing at least a portion of the body part to be accommodated wherein
the cushion blank is subdivided into a calf part and a food part, whereby the foot part is designed to be T-shaped, with a sole part assigned to the sole of the foot and a heel part which runs across the longitudinal axis of the sole part and is divided from the sole part by a heel constriction, with foot side parts extending on both sides of the longitudinal axis.

3. The molded cushion according to claim 2, characterized in that
the foot side parts have lower edges running essentially perpendicular to the longitudinal axis of the sole part and upper edges running to the free side part ends with an inclination toward the lower edges.

4. The molded cushion according to claim 2, characterized in that
the cushion chamber is designed like a frame in the area of the foot side parts with a recess surrounded by the cushion chamber on all sides.

5. The molded cushion according to claim 2, characterized in that
the heel part is provided with a recess in a heel area.

6. The molded cushion according to claim 2, characterized in that
the cushion chamber has a U-shaped cushion area in the area of the calf part with a chamber base and two chamber legs, with a leg connection being provided between the chamber legs.

7. The molded cushion casing for forming an orthotic inlay, characterized in that
the molded cushion has a planar cushion blank with a base part with at least two side parts and a strap part which is separated from the base part by a constriction, and the molded cushion casing has at least three receptacle areas assigned to the side parts of the base part and the strap part, namely two side part receptacles and one strap part receptacle, which are arranged adjacent to one another such that after being accommodated in the respective receptacle areas of the molded cushion casing the side parts and the strap part of the molded cushion form a shell-like body part receptacle for enclosing at least a portion of the body part to be accommodated, wherein
the molded cushion casing has connecting seams which join a lower layer and an upper layer to form and separate the receptacle areas.

8. The molded cushion cashing for forming an orthotic inlay for arrangement on a lower leg, characterized in that
the molded cushion has a planar cushion blank with a base part with at least two side parts and a strap part which is separated from the base part by a constriction, and the molded cushion casing has at least three receptacle areas assigned to the side parts of the base part and the strap part, namely two side part receptacles and one strap part receptacle, which are arranged adjacent to one another such that after being accommodated in the respective receptacle areas of the molded cushion casing the side parts and the strap part of the molded cushion form a shell-like body part receptacle for enclosing at least a portion of the body part to be accommodated wherein
the molded cushion casing has a calf part receptacle and a foot part receptacle to accommodate a calf part and a foot part of the molded cushion, whereby the foot part receptacle has a sole pocket to accommodate a sole part with two side part pockets running essentially parallel and adjacent to the former to accommodate the foot side parts.

9. A valve mechanism for a molded cushion of an orthotic inlay wherein a molded cushion has a planar cushion blank with a base part with at least two side parts and a strap part which is separated from the base part by a constriction, and the molded cushion casing has at least three receptacle areas assigned to the side parts of the base part and the strap part, namely two side part receptacles and one strap part receptacle, which are arranged adjacent to one another such that after being accommodated in the respective receptacle areas of the molded cushion casing the side parts and the strap part of the molded cushion form a shell-like body part receptacle for enclosing at least a portion of the body part to be accommodated, the valve mechanism having a valve connection made of an elastically deformable plastic material for joining to the airtight casing of the molded cushion and a valve insert accommodated in the valve connection to accommodate a membrane disk which seals the valve cross section of the valve insert, characterized in that
a clamping ring is provided on the vale connection, which clamping ring is displaceable in the longitudinal direction of the valve connection and is convertible from a sealing position in which the membrane disk is essentially in flat contact with a sealing edge of the valve insert into a venting position in which the membrane disk is deformed by the deformed valve connection.

10. The valve mechanism according to claim 9, characterized in that
the valve insert is designed like a sleeve with a membrane guide arranged concentrically with the sealing edge of the valve insert to accommodate a guidance journal of the membrane disk.

11. The molded cushion according to claim 3, characterized in that
the cushion chamber is designed like a frame in the area of the foot side parts with a recess surrounded by the cushion chamber on all sides.

12. The molded cushion according to claim 3, characterized in that
the heel part is provided with a recess in a heel area.

13. The molded cushion according to claim 4, characterized in that
the heel part is provided with a recess in a heel area.

14. The molded cushion according to claim 11, characterized in that
the heel part is provided with a recess in a heel area.

15. The molded cushion according to claim 3, characterized in that
the cushion chamber has a U-shaped cushion area in the area of the calf part with a chamber base and two chamber legs, with a leg connection being provided between the chamber legs.

16. The molded cushion according to claim 4, characterized in that
the cushion chamber has a U-shaped cushion area in the area of the calf part with a chamber base and two chamber legs, with a leg connection being provided between the chamber legs.

17. The molded cushion according to claim 11, characterized in that
the cushion chamber has a U-shaped cushion area in the area of the calf part with a chamber base and two chamber legs, with a leg connection being provided between the chamber legs.

18. The molded cushion according to claim 5, characterized in that
the cushion chamber has a U-shaped cushion area in the area of the calf part with a chamber base and two chamber legs, with a leg connection being provided between the chamber legs.

19. The molded cushion according to claim 12, characterized in that
the cushion chamber has a U-shaped cushion area in the area of the calf part with a chamber base and two chamber legs, with a leg connection being provided between the chamber legs.

20. The molded cushion according to claim 13, characterized in that
the cushion chamber has a U-shaped cushion area in the area of the calf part with a chamber base and two chamber legs, with a leg connection being provided between the chamber legs.

21. The molded cushion according to claim 14, characterized in that
the cushion chamber has a U-shaped cushion area in the area of the calf part with a chamber base and two chamber legs, with a leg connection being provided between the chamber legs.
